(19) United States
(12) Patent Application Publication
(10) Pub. No.: US 2016/0174961 A1
(43) Pub. Date: Jun. 23, 2016

(54) METHODS OF SOFT TISSUE FIXATION USING FILAMENTARY TISSUE ANCHORS

(71) Applicant: Howmedica Osteonics Corp., Mahwah, NJ (US)

(72) Inventors: Ross Callison, Denver, CO (US); Benjamin Yore, Castle Rock, CO (US); José Raúl Marchand, San Juan, PR (US); Keith Taylor, Parker, CO (US); Jaime Genuario, Lone Tree, CO (US)

(21) Appl. No.: 14/573,538

(22) Filed: Dec. 17, 2014

(51) Int. Cl. A61B 17/04; A61B 17/06

(52) U.S. Cl. CPC A61B 17/0401; A61B 17/06166; A61B 2017/0409; A61B 2017/0414

(57) ABSTRACT

A method of tissue fixation, which includes inserting a sleeve into a bone hole. The sleeve includes first and second openings and a length of filament slidably disposed therethrough. The length of filament includes a first free end extending from the first opening of the sleeve and a loop-end extending from the second opening of the sleeve. The method also includes tensioning the loop-end and first free end to secure the sleeve in the bore hole, passing the first free end through tissue at a first location and passing the loop-end through tissue at a second location, and advancing the first free end through a loop defined by the loop-end.

### BACKGROUND OF THE INVENTION

Surgical repair of soft tissue often requires damaged soft tissue or replacement graft tissue to be positioned against adjacent soft tissue or hard tissue (e.g., bony structure). The objective is to form a healing interface so that microscopic connections can be formed during the healing process, thereby adjoining the contacting tissue structures. In order to achieve this objective, it is important to maintain and minimize disruptions at this interface. Otherwise these connections and ultimately the entire repair can be compromised.

In one example, a portion of torn tissue that is typically connected to a bony structure, such as a labrum, rotator cuff, Achilles tendon, patellar tendon, or the like may be connected or reconnected to the bony structure. This is typically achieved by positioning the torn tissue as close to its natural location as possible and anchoring the tissue to the bone. Compression between the bone and tissue is desirable to help maintain the healing interface and to instigate the healing process.

Generally, an anchoring support and a filament attached to the anchoring support are utilized in soft tissue reparation. A surgical knot is typically created to hold the tissue against the bone. However, these surgical knots are subject to loosening, which can reduce or eliminate desirable compression and can lead to undesirable movement of the healing interface, which may result in a suboptimal repair or total failure of the repair.

Despite the use and benefits of such devices and techniques, such devices and techniques can benefit from alternative devices and securement techniques.

### BRIEF SUMMARY OF THE INVENTION

In one aspect of the disclosure, a method of tissue fixation includes inserting a sleeve into a bone hole. The sleeve has first and second openings and a length of filament slidably disposed therethrough. The length of filament has a first free end extending from the first opening of the sleeve and a loop-end extending from the second opening of the sleeve. The method also includes tensioning the loop-end and first free end to secure the sleeve in the bore hole. Further included in the method is passing the first free end through tissue at a first location and passing the loop-end through tissue at a second location. Additionally, the method includes advancing the first free end through a loop defined by the loop-end.

Additionally, the method may include compressing tissue disposed between the first location and second location against bone by tensioning the first free end. Further, the method may also include passing a second free end of the length of filament through the tissue at a third location, and advancing the second free end through the loop. The first free end of the length of filament may include first and second portions each having a cross-section defining a height and a width. The width of the first portion may be greater than the width of the second portion. After the advancing step, the first portion may extend into and through the loop and the second portion may extend away from the loop. The loop-end may be formed by folding the single length of filament at a location along its length. Alternatively, the loop-end may be formed by splicing the single length of filament with itself at a location along its length.

Continuing with this aspect, passing the loop-end through the second location may include passing a second free end of the length of filament through the tissue at the second location. The loop may include a crotch and an apex. After the advancing step, the first free end may extend through the loop at the apex and the second free end may extend from the crotch.

In another aspect of the disclosure, a method of tissue fixation includes inserting an anchor into a bone hole. The anchor has a length of filament slidably disposed therein. The length of filament is comprised of a first portion and a second portion. The first portion has a cross-sectional dimension larger than a cross-sectional dimension of the second portion. The second portion forms a loop. The method also includes passing the first portion through tissue at a first location and the loop at least partially through tissue at a second location. The method further includes advancing the first portion through the loop.

Additionally, the anchor may be a filamentary sleeve that has a first opening and a second opening in which the first portion may extend from the first opening and the loop may extend from the second opening. The loop may be formed by splicing the single length of filament with itself at a location along its length. The first portion may include a flat cross-sectional profile that has a width and a height, and the second portion may include a round cross-sectional profile that has a diameter. Alternatively, the first portion may include a flat cross-sectional profile that has a width and a height, and the second portion may include a rectangular cross-sectional profile that has a width and a height.

Continuing with this aspect, the method may include passing a third portion of the length of filament through the tissue at the second location. The third portion may extend from a crotch of the loop and may have a cross-sectional dimension larger than the cross-sectional dimension of the second portion.

The method may also include passing a third portion of the length of filament through the tissue at a third location. The third portion may have a cross-sectional dimension larger than the cross-sectional dimension of the second portion. The third portion may be advanced through the loop.

In a further aspect of the present disclosure, a method of tissue fixation includes obtaining an insertion device having a head connector, a first head having a first filamentary sleeve coupled thereto, a second head having a second filamentary sleeve coupled thereto, and a single length of filament extending through the first and second sleeves. The method also includes inserting the first head and first filamentary sleeve into bone at a first location. With the first sleeve remaining in the first location, the first head is removed from the bone. The first head is removed from the head connector. The method also includes attaching the second head to the head connector, inserting the second head and second filamentary sleeve into the bone at a second location, and removing the second head from the second head connector. Within the method, the first and second filamentary sleeves are disposed in the bone and the single length of filament is disposed at least partially within the first and second filamentary sleeves.

Additionally, a first free end of the single length of filament may extend from the first filamentary sleeve and a second free end of the single length of filament may extend from the second filamentary sleeve. Also in the method, the inserting step may include inserting the first and second heads through tissue such that a segment of the single length of filament extending between the first and second sleeves compresses the tissue against the bone. Inserting the first and second filamentary sleeves may occur concurrently.

Continuing with this aspect, the first and second free ends of the single length of filament may extend from the first filamentary sleeve and a loop-end of the single length of filament may extend from the second filamentary sleeve. The method may also include passing the first and second free ends through tissue at first and second locations respectively, passing the loop-end through the tissue at a third location, and advancing the first and second free ends through a loop defined by the loop-end.

In a still further aspect of the present disclosure, a method of tissue fixation includes inserting a first head of an insertion device through tissue and into a first bone hole. The first head may be attached to a connector and have a first sleeve attached thereto. The insertion device may also have a second head having a second sleeve attached thereto and a third head may also have a third sleeve attached thereto. A length of filament may be slidably disposed through the first, second, and third sleeves. The method may include securing the first sleeve within the first bone hole while the length of filament remains slidably disposed within the first, second, and third sleeves. The method may also include removing the first head from a connector, attaching the second head to the connector, inserting the second head through the tissue and into a second bone hole, and securing the second sleeve within the second bone hole.

Continuing with this aspect, the method may include removing the second head from the connector, attaching the third head to the connector, inserting the third head through tissue into a third bone hole, and securing the third sleeve within the third bone hole. Additionally, the method may include tensioning the length of filament, connecting an end of the length of filament to a bone anchor, and anchoring the bone anchor into the bone.

### BRIEF DESCRIPTION OF THE DRAWINGS

The features, aspects, and advantages of the present invention will become better understood with regard to the following description, appended claims, and accompanying drawings in which:

FIG. 1A illustrates one embodiment of a tissue fixation assembly.

FIGS. 1B and 1C are schematic side views of a configuration of the fixation assembly of FIG. 1A.

FIGS. 1D and 1E are schematic top views of the configuration of the fixation assembly of FIG. 1A.

FIG. 2A illustrates another embodiment of a tissue fixation assembly.

FIGS. 2B and 2C are schematic side views of a configuration of the tissue fixation assembly of FIG. 2A.

FIG. 2D is a schematic top view of the configuration of the fixation assembly of FIG. 2A.

FIG. 3A illustrates a further embodiment of a tissue fixation assembly.

FIGS. 3B and 3C are schematic side views of a configuration of the tissue fixation assembly of FIG. 3A.

FIGS. 3D and 3E are schematic top views of the configuration of the tissue fixation assembly of FIG. 3A.

FIG. 4A illustrates yet another embodiment of a tissue fixation assembly.

FIG. 4B is a cross-sectional schematic view of the tissue fixation assembly of FIG. 4A taken at A-A.

FIG. 4C is a schematic side view of a configuration of the tissue fixation assembly of FIG. 4B.

FIGS. 4D and 4E are schematic top views of the configuration of the tissue fixation assembly of FIG. 4A.

FIG. 5A illustrates still another embodiment of a tissue fixation assembly that includes a sleeve and a length of filament.

FIG. 5B illustrates exemplary braiding patterns of the length of filament of FIG. 5A.

FIG. 5C is a schematic side view of a configuration of the tissue fixation assembly of FIG. 5A.

FIGS. 5D and 5E are schematic top views of a first arrangement of the tissue fixation assembly of FIG. 5A and configuration of FIG. 5C.

FIG. 6A illustrates yet a further embodiment of a tissue fixation assembly.

FIG. 6B illustrates one embodiment of an inserter device.

FIG. 6C is a schematic side view of an exemplary configuration of the tissue fixation assembly of FIG. 6A employing inserter device of FIG. 6B.

FIGS. 6D and 6E are schematic top views of the configuration of the tissue fixation assembly of FIGS. 6A and 6C.

FIG. 7 illustrates an alternative embodiment inserter device.

### DETAILED DESCRIPTION

The fixation devices, assemblies, systems, and associated methods of use of the present invention are intended for use in the repair, reattachment, replacement, or otherwise securement of tissue, including both hard tissue (e.g., bone or the like) and soft tissue. Soft tissue may be, for example, meniscus, cartilage, capsule, ligaments and tendons, replacement grafts of any of these soft tissues, or the like. While many of the exemplary methods disclosed herein are directed towards the use of fixation assemblies, systems, and methods involving a filament/suture anchor for implantation into a bone hole, it is envisioned that such assemblies, systems, and methods described herein can be utilized with a hard/solid anchor in lieu of or in conjunction with a filament/suture anchor. In addition, it should be understood that the following devices and methods may be utilized in both open surgery and arthroscopic surgery.

As used herein unless stated otherwise, the term “anterior” means toward the front part of the body or the face, the term “posterior” means toward the back of the body. The term “medial” means closer to or toward the midline of the body, and the term “lateral” means further from or away from the midline of the body. In addition, the terms “about,” “generally” and “substantially” are intended to mean that slight deviations from absolute are included within the scope of the term so modified.

Also, as used herein, the term “filament” or “filamentary” is defined as a suture or other thread-like material. Such filaments may be constructed of synthetic material (e.g., PLGA, UHMWPE (ultra high molecular weight polyethylene), polyester, PEEK, nylon, polypropylene, aramids (for example Kevlar®-based fibers) or the like, or blends thereof), organic material (silk, animal tendon, or the like or blends thereof), or blends of both one or more organic materials and one or more synthetic materials. Alternatively, filaments may include thin metal wires. While any of these materials may be used, it is preferable, and is disclosed herein, that the various filaments or filamentary aspects of the present invention be constructed out of suture, such as UHMWPE, polyester or blends thereof.

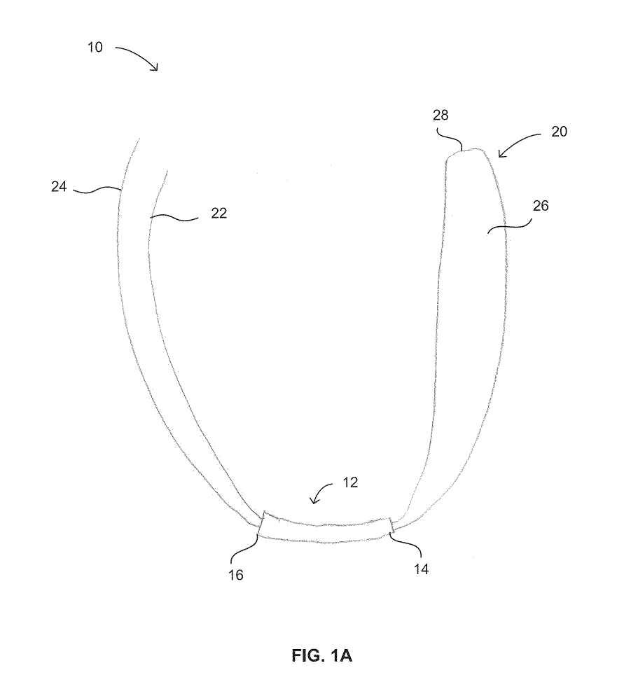

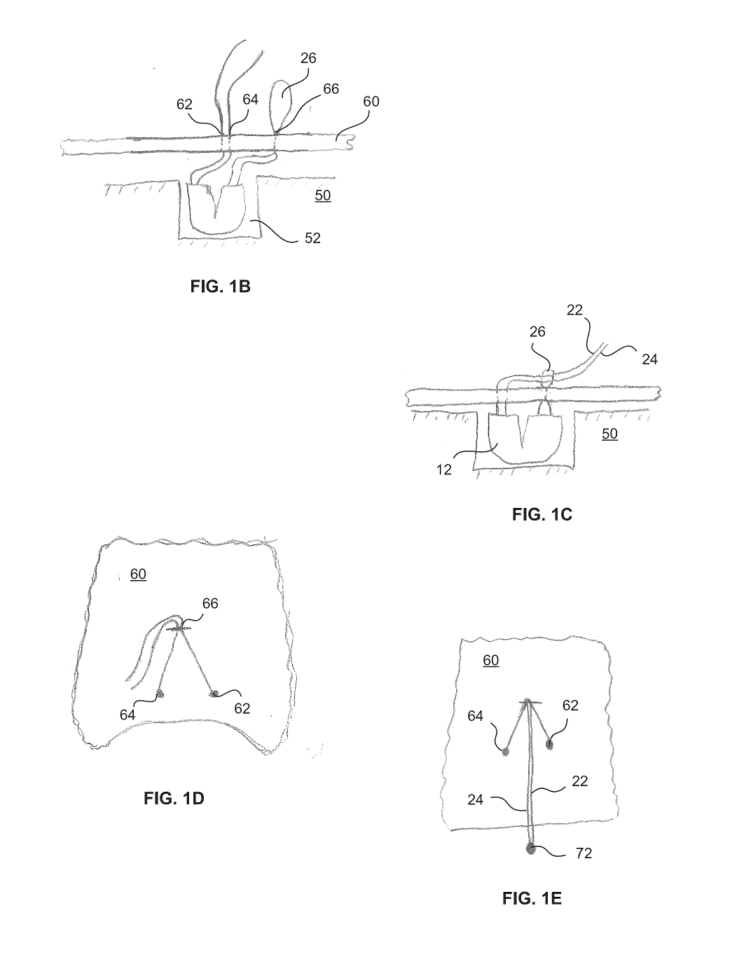

FIG. 1A depicts an embodiment of a fixation assembly 10. Fixation assembly 10 includes a filamentary sleeve 12 and a length of filament 20. Sleeve 12 includes a first opening and a second opening 16 and a passageway extending therethrough. In one example, the sleeve 12 can be the Iconix® all suture anchor system (Stryker Corporation, Kalamazoo, Mich.). Other configurations are also envisioned, examples of which are disclosed in U.S. application Ser. No. 13/783,804, filed Mar. 4, 2013; Ser. No. 13/303,849, filed Nov. 23, 2011; Ser. No. 13/588,586, filed Aug. 17, 2012; Ser. No. 13/588,592, filed Aug. 17, 2012; Ser. No. 14/104,677, filed Dec. 12, 2013; Ser. No. 14/298,295, filed Jun. 6, 2014; and U.S. Pat. Nos. 5,989,252 and 6,511,498, the entireties of which are incorporated by reference herein as if fully set forth herein and all of which are assigned to the same entity as the present invention.

Filament 20 is folded over itself at a location along its length to form a loop 26 that defines a loop-end of filament 20 and an apex 28. Filament 20 is disposed at least partially within the passageway of the sleeve 12 such that the loop-end extends from one end of sleeve 12 and first and second free ends 22 and 24 of filament 20 extend from the opposite end of sleeve 12.

FIGS. 1B-1D depict an embodiment method of using fixation assembly 10. This method may be utilized in many procedures in which soft tissue is to be attached or otherwise anchored to bone. For ease of illustration, the various disclosed exemplary methods and uses throughout will be described with reference to a rotator cuff repair, though these methods and uses may be translated to other soft tissue repairs. In such a method, a bone hole 52 is drilled or otherwise formed in bone 50. An inserter device (not shown) may be attached to sleeve 12 such that sleeve 12 and inserted into bone hole 52 as shown in FIGS. 1B and 1C. At this point, the loop-end and first and second free ends 22 and 24 extend from bone hole 52 and are tensioned to seat sleeve 12 into an anchoring position within bone hole 52.

With the loop-end and free ends 22, 24 extending from bone hole 52, first free end 22 is passed through tissue 60 at a first tissue penetration location 62, second free end 24 is passed through a second tissue penetration location 64, and the loop-end is passed through a third tissue penetration location 66. In some embodiments, free ends 22 and 24 may be passed through the same tissue penetration location. Free ends 22 and 24 are passed through loop 26 and continuously tensioned until loop 26 cinches down around free ends 22 and 24. As loop 26 is cinched, tissue 60 is drawn closer to and compressed against bone 50 surrounding hole 52.

Free ends 22 and 24 are available to be utilized in conjunction with at least one additional anchor (filamentary or the like), for example, in the formation of a suture bridge. As such, no knots need be formed and continuous tension may be applied to free ends 22 and 24 keeping loop 26 cinched and tissue 60 compressed against bone 50.

The tissue penetration locations 62, 64 and 66 can be arranged in any number of configurations and may generally form a triangular pattern as in FIG. 1D. For example, the first, second and third penetrations 62, 64, 66 can be situated to form an equilateral triangle. In another embodiment, an isosceles triangle may be formed in which first and second penetrations 62 and 64 are substantially equally spaced from third penetration 66. In a further embodiment, penetration 62, 64, 66 may be arranged in the form of a right triangle such that first or second penetration is closer to the third penetration 66 than the other penetration. Other triangular configurations may also be utilized. In addition, penetration locations 62, 64, 66 may be located all within an area directly above bore hole 52, or one or more penetration may be located beyond the periphery of bore hole 52.

In some circumstances, a particular triangular configuration may be chosen to help direct tension applied to the tissue via filament 20. For example, as depicted in FIG. 1D, tissue 60 may be a rotator cuff. Third penetration 66, as depicted, is located medial of first and second penetrations 62 and 64, which are aligned in a row in an anterior/posterior direction. First and second penetrations 62 and 64 are equally spaced from third penetration 66 to form an isosceles or equilateral triangular pattern. Tension is applied to free ends 22 and 24 in either the lateral or medial direction, which cinches loop 26. As loop 26 becomes tighter, the resultant tension applied to the rotator cuff 60 is in a direction which substantially bisects first and second penetrations 62, 64, which is at least partially due to the symmetrical nature of the depicted triangular configuration. Thus, in the example provided in FIG. 1E, the rotator cuff 60 would be tensioned in substantially the medial/lateral direction toward the humerus. Free ends may then be directed over apex and fixed to a second and/or third bone anchor 72 that is disposed lateral to sleeve 12.

In another example, a right-triangular pattern may be formed in which first penetration 62 is closer to third penetration 66. When free ends 22 and 24 are advanced through loop 26 and tensioned, the resultant tension applied to the rotator cuff may be in both the lateral/medial and anterior/posterior directions.

It should be understood that a triangular configuration comprised of penetrations 62, 64 and 66 may have alternative orientations from that depicted in FIG. 1D. For instance, in one embodiment, the triangular pattern shown in FIG. 1D may be mirrored such that third penetration 66 is located lateral to first and second penetrations 62, 64. Alternatively, the triangular pattern may be oriented orthogonally from the depicted location such that third penetration 66 is located more anteriorly or more posteriorly than first and second penetrations 62, 64. The specific orientation and positioning of the penetration locations (indeed, the configuration of any of the disclosed devices and methods herein) may be dependent on the type of repair required. For example, for the rotator cuff, such positioning can be dependent on whether the injury is a full thickness, partial thickness, PASTA lesion, trans-tendinous, or the like.

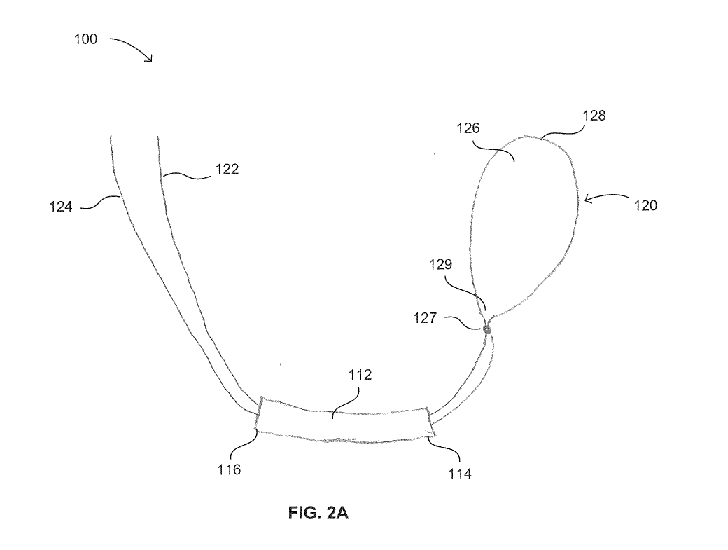

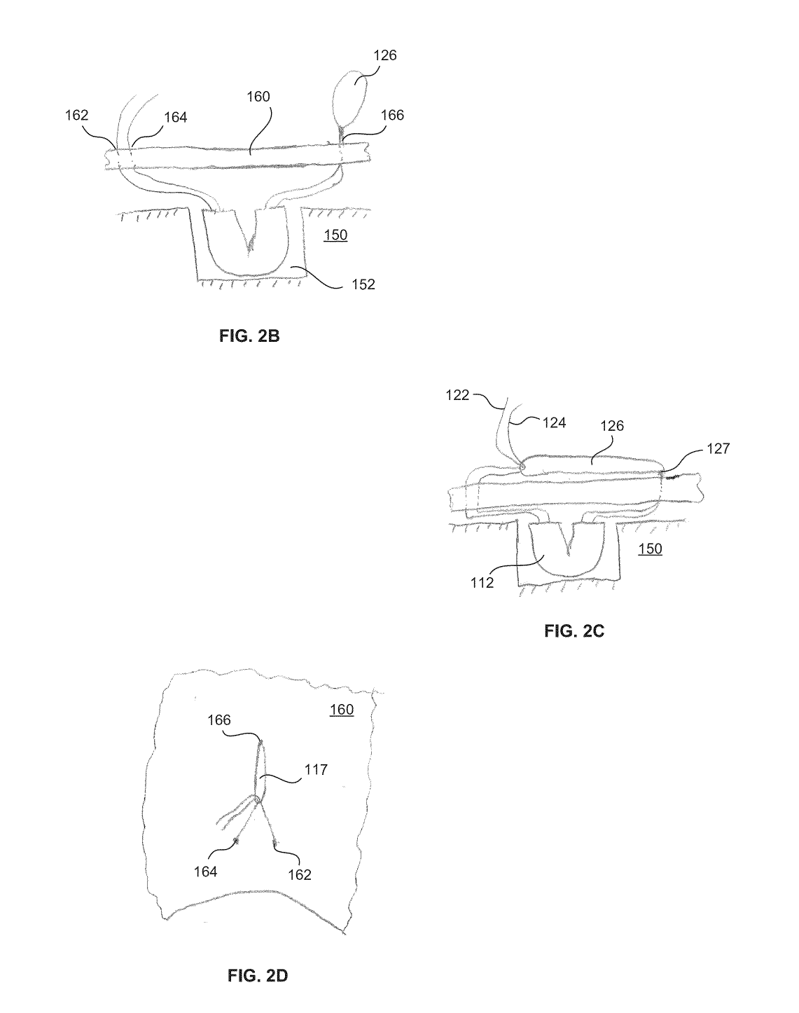

FIG. 2A depicts an alternative embodiment fixation assembly 100. Fixation assembly 100 is similar to fixation assembly 10 in that it includes a filamentary sleeve 112 and length of filament 120, which can be the same as filamentary sleeve 12 and length of filament 20, respectively. However, fixation assembly 100 differs in that filament 120 is joined at a junction 127 to form a loop 126 that defines a loop-end of filament 120, an apex 128, and a crotch 129. Junction 127 may be formed by a splice, such as a Brummel splice, by braiding filament 120 together at the junction location, by mechanical means, such as a clamp, or by some other means as is known in the art.

FIGS. 2B-2D depict a method of using fixation assembly 100. The method of using fixation assembly 100 is similar to the method of using fixation assembly 10 in that sleeve 112 is inserted and anchored into a bone hole 152. Thereafter, first and second free ends 122, 124 of filament 120 are passed through tissue 160 at first and second tissue penetration locations 162, 164, respectively, while the loop-end is passed through a third tissue penetration location 166. Such penetration locations 162, 164, 166 may be arranged in various triangular patterns as previously described.

As shown in FIGS. 2B-2D, at least a portion of loop 126, and optionally all of the loop 126 and junction 127, is passed through tissue 160. In particular, as illustrated, when free ends 122 and 124 are tensioned, loop 126 remains above the tissue without reentering penetration 166. Junction 127 may be configured such that it collapses over penetration 166 or is otherwise structured so that it cannot be passed back through tissue 160. For example, junction 127 can have braiding or an additional sleeve/skirt attached to junction 127 that has a narrow profile while passing through tissue 160 in one direction, while collapsing and expanding when there is an attempted advancement back through tissue 160. In another example, a thermoreactive material, such as hydrogel or Nitinol can be applied at the location such that it expands upon the application of heat once passed through tissue 160. Such a configuration may assist in compression of the tissue, at location 166, against underlying bone.

In another embodiment, loop 126 and junction 127 may not completely exit penetration 166 or may be readily passed back into and through penetration 166. Thus, as free ends 122 and 124 are tensioned, a portion of loop 126 is pulled into sleeve 112 as another portion of loop 126 cinches down around free ends 122 and 124 resulting in a configuration that has the appearance of FIG. 1D.

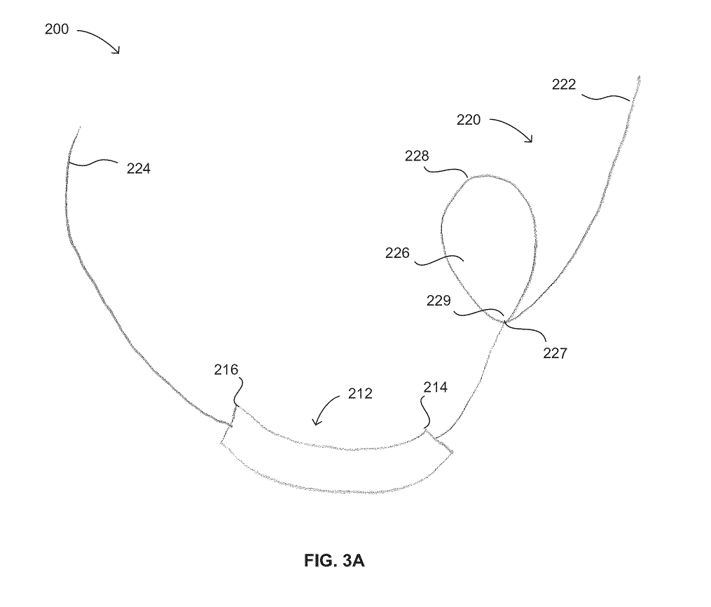

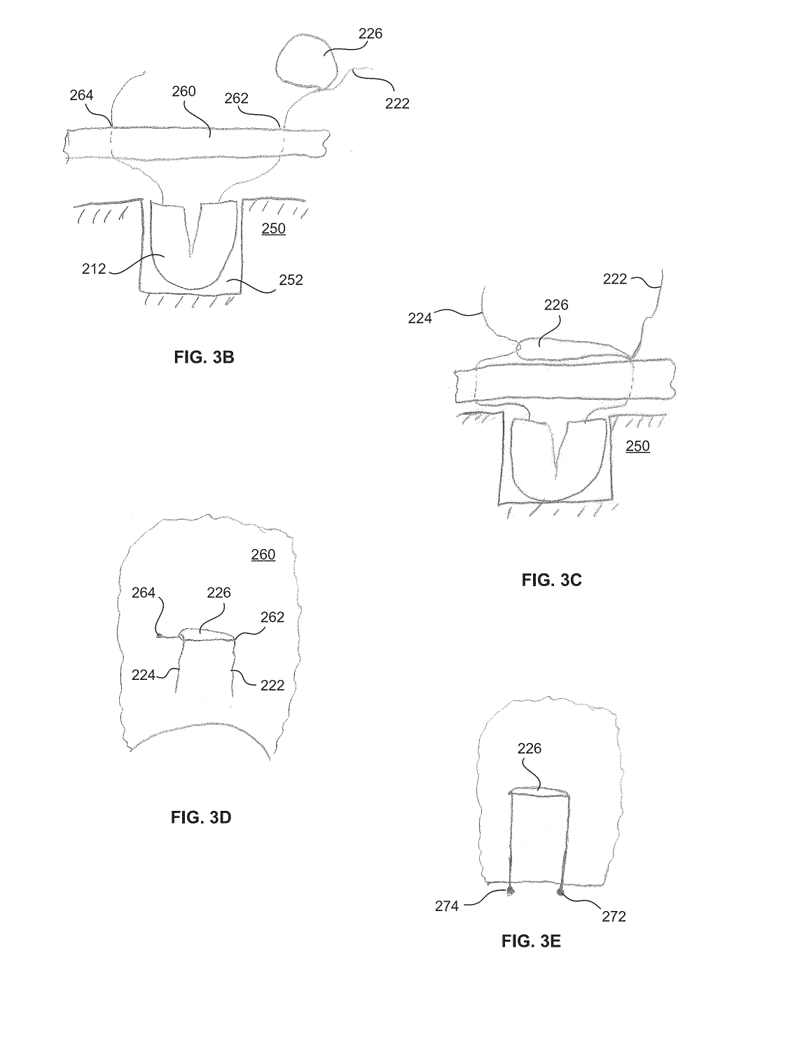

FIG. 3A depicts another fixation assembly embodiment 200. Fixation assembly 200 also includes a filamentary sleeve 212 and length of filament 220. Sleeve 212 may be the same as filamentary sleeve 12. Filament 220 may be similar to filament 120 such that filament 220 includes a junction forming a loop 226 that defines a loop-end of filament 220, a crotch 229 and an apex 228. Filament 220 also includes a first free end 222 and a second free end 224 that each extend from the junction. In some embodiments, first free end 222 may have a shorter length than second free end 224, a longer length than free end 224 or the same length as free end 224, but, regardless, both may have a length sufficient to be used in conjunction with an arthroscopic cannula. When sleeve 212 and filament 220 are assembled, second free end 224 passes through the passageway of sleeve 212 and out of the second end 216, while loop 226 and first free end 222 extend from the first end 214 of sleeve 212.

FIGS. 3B-3E depict one exemplary method embodiment of using fixation assembly 200. As shown in FIGS. 3B and 3C, a bone hole 252 is formed in bone 250, and filamentary sleeve 212 is inserted and anchored into bone hole 252. First free end 222 and loop 226 are passed through a first tissue penetration location 262, and second free end is passed through a second tissue penetration location 264. Thereafter, second free end 224 is advanced through loop 226 and tensioned. First free end 222 may also be tensioned simultaneously with the second free end 224 to help tension the structure and to help prohibit loop 226 and first free end 222 from being drawn through second penetration 262 during tensioning of second free end 264. As tension is applied, filament 220 compresses tissue 260 against bone 250.

As shown in FIGS. 3D and 3E, tissue 260 may be a rotator cuff, and first and second penetrations 262, 264 may be aligned in an anterior/posterior direction. Second free end 224 extends from loop 226 at apex 228, first free end 222 extends from junction 227, and loop 226 extends along tissue 260 between first and second penetrations 262, 264. Thereafter, first and second free ends 222, 224 may be secured laterally to bone 250 via bone anchors 272 and 274, respectively. Bone anchors 272 and 274 may each be a filamentary anchor, such as sleeve 12, or a solid anchor as is known in the art. Alternatively, both the first and second free ends 222, 224 may be secured laterally to bone 250 via a single bone anchor (not shown). Therefore, as described, a surgical knot need not be applied.

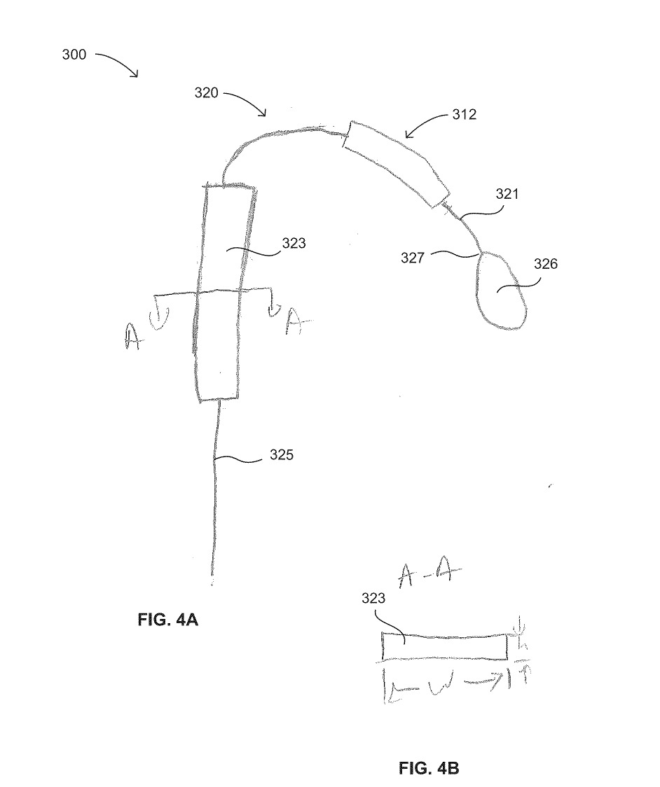

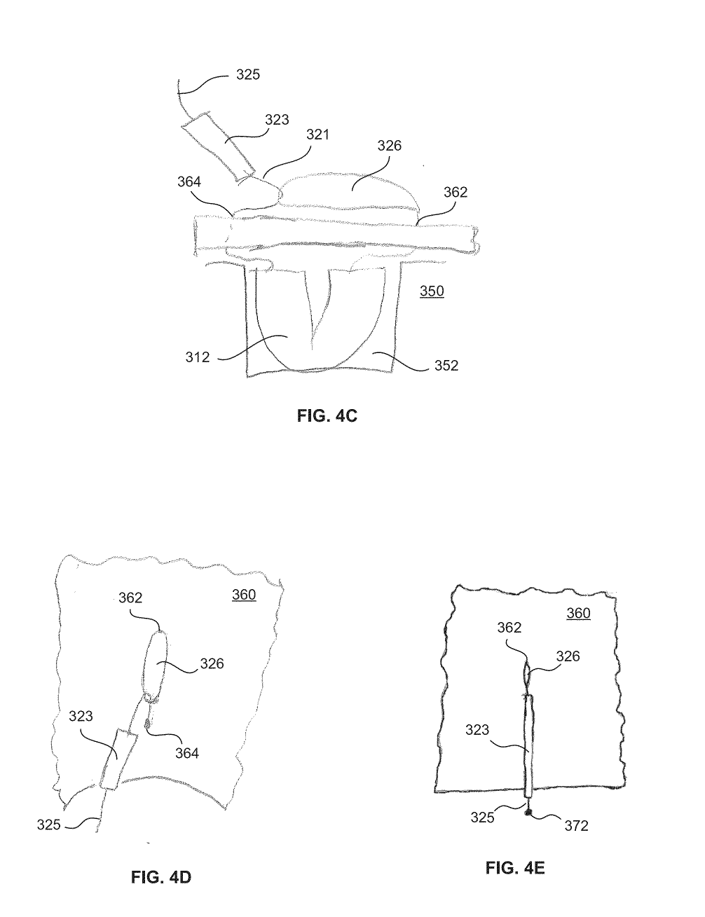

FIG. 4A illustrates another fixation assembly embodiment, fixation assembly 300. Fixation assembly 300 includes a filamentary sleeve 312 and a length of filament 320. Filamentary sleeve 312 may be the same as sleeve 12.

Filament 320 includes a first end 321, a second end 325, and a tape portion 323 disposed between first and second ends 321, 325. First and second ends 321, 325 are joined to tape portion 323 either by being braided together as a single construct or are coupled by other means such as gluing, sewing, or welding together, for example. First end 321 of filament 320 includes a loop 326. Loop 326 may be formed as previously described, for example, by splicing filament 320 at junction 327 to form loop 326.

Tape portion 323 has a generally flat cross-section that includes a height (h) and width (w), as shown in FIG. 4B. First and second ends 321, 325 preferably include a rounded cross-sectional profile having a diameter. Thus, as shown, filament 320 may have a round-flat-round configuration. The width of the tape portion 323 is preferably greater than the diameter of the first and second ends 321, 325, while the height of the tape portion 323 may be substantially equal to or less than the diameter of ends 321 and 325.

In one embodiment, filament 320 may have a round-flat configuration in which filament 320 would only be comprised of end 321 and flat portion 323. In such an embodiment, end 321 would form loop 326. In another embodiment, filament 320 may have a flat-round configuration in which filament 320 would only be comprised of flat portion 323 and end 325. In this embodiment, tape portion 323 would form loop 326. In a further embodiment, filament 320 may be flat along its entire length. In other words, in this example filament 320 may be comprised entirely of tape portion 323 with no rounded portions/ends. In yet another embodiment, first and second ends 322, 323 may have a rectangular cross-sectional profile in which the width of tape portion 323 may be greater than the width of the ends 321 and 325, and the height of the tape portion may be substantially equal to or less than the height of the tape portion. The flat profile and relatively large width of the tape portion may facilitate a broad compressive footprint and help reduce irritation of the tissue. Such filaments may have any configuration of round and/or flat portions as desired.

When assembled, sleeve 312 is preferably arranged about first end 321 such that first end 321 is at least partially disposed within the passageway of sleeve 312. In the embodiment shown in FIG. 4A, or other embodiments, such as a flat-round embodiment or entirely flat embodiment, sleeve 312 may be alternatively arranged about tape portion 323.

FIGS. 4C-4E depict an exemplary method of using fixation device 300. A bone hole 352 is formed in bone 350, and sleeve 312, which is slidably attached to first end 321, is inserted into bone hole 352, as shown in FIG. 4C. First and second ends 321, 325 are tensioned to seat sleeve 312 into an anchoring position. Loop 326 is at least partially passed through a first tissue penetration location 362, and second end 325 and tape portion 323 are passed through a second tissue penetration location 364. Second end 325 and tape portion 323 are advanced through the loop 326 such that loop 326 encompasses a portion of first end 321.

In an example of a rotator cuff, as illustrated in FIGS. 4D and 4E, second end 325 is tensioned and tape portion 323 is extended over tissue 360. Second end 325 is then attached to a bone anchor (filamentary or the like) and secured to bone 350. In this manner, tape portion 323 forms a broad compressive footprint to facilitate tissue adhesion to bone 350.

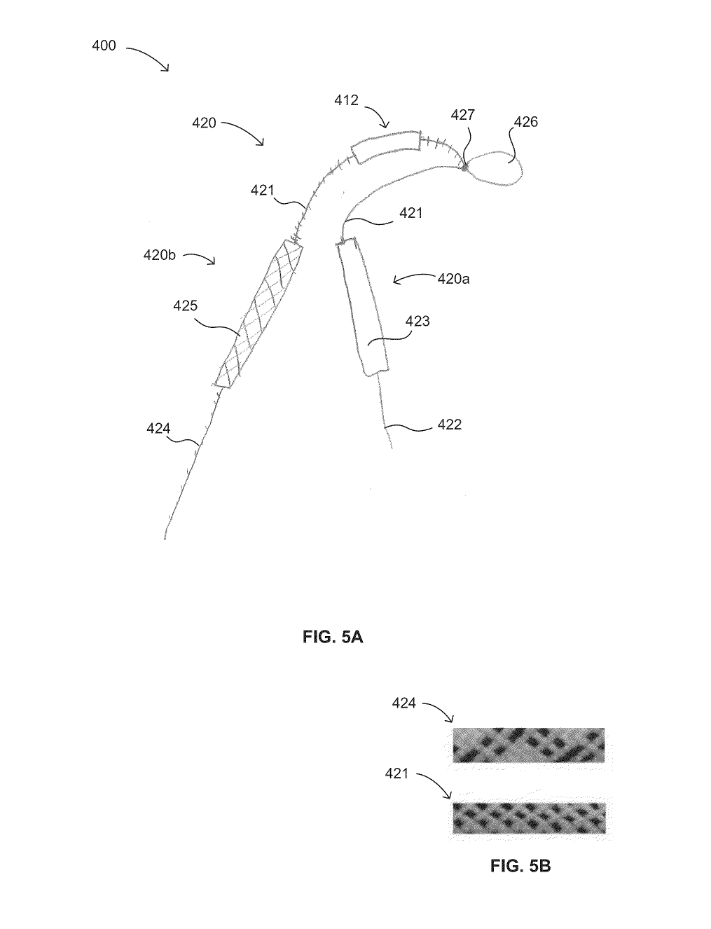

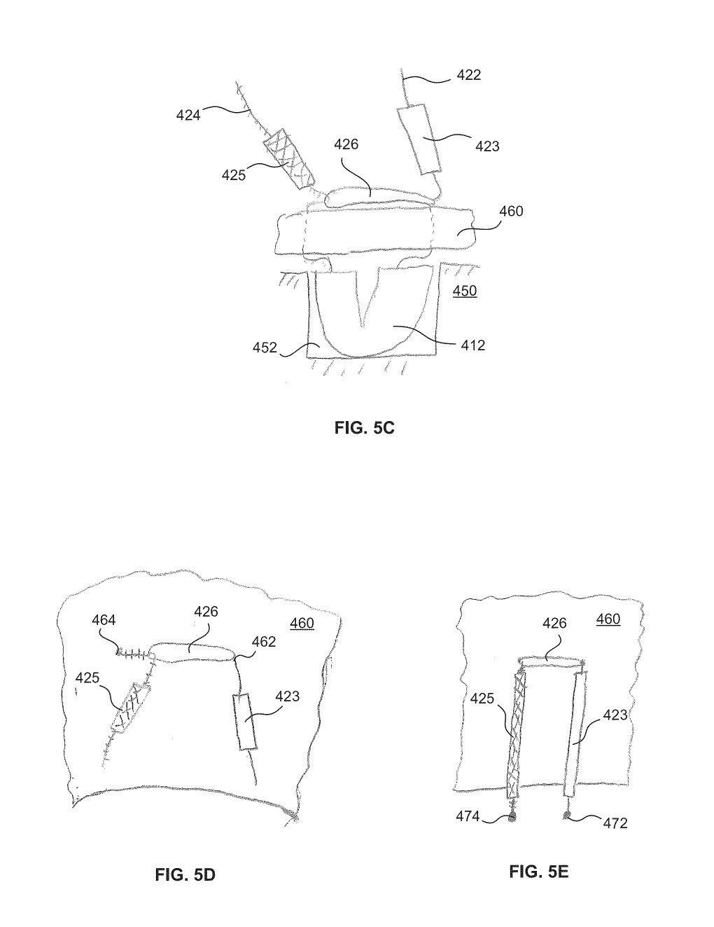

FIG. 5A depicts a further fixation assembly embodiment, fixation assembly 400. Fixation assembly 400 includes a filamentary sleeve 412 and a length of filament 420. Filamentary sleeve 412 may be the same as sleeve 12.

Filament 420 is divided into a first segment 420a and a second segment 420b each having a distinctive braiding pattern. For example, first segment may have spiral braiding pattern 424, and second segment may have a speckled braiding pattern 421, as shown in FIG. 5B. However, it should be understood that filament 420 can have the same braiding pattern throughout, or a pattern along only one segment or along a portion of one or both segments, or the like.

The braiding pattern or patterns may be formed in any manner desired. For example, one or more fibers of a distinct color may be woven into the braid (as in FIG. 5B) to create a desired pattern along a portion, segment or the entirety of the filament. In another example, a surgical marker or pen may be used to mark a portion, segment or the entirety of the filament with a particular pattern, color or the like. For instance, a blue pen could be used to designate segment 420a while a red pen could be used to designate segment 420b. Such pattern or color differences can assist a surgeon in keeping track of the filament lengths during the surgical procedure.

Filament 420 includes a first end portion 422, a second end portion 424, an intermediate portion 421, a first tape portion 423, and a second tape portion 425. First tape portion 423 is disposed between first end 422 and intermediate portion 421, and the second tape portion 425 is disposed between the second end 424 and intermediate portion 421. A loop 426 is formed by intermediate portion 421, for example, by splicing filament 420 at a junction 427.

First segment 420a comprises loop 426, first end 422, first tape portion 423, and a length of intermediate portion 421 that extends from the junction 427 to the first tape portion 423. Second segment 420b comprises second end 424, second tape portion 425, and a length of intermediate portion 421 that extends from junction 427 to second tape portion 425.

Tape portions 423 and 425 are similar to tape portion 323. First end 422, second end 424, and intermediate portion 421 are similar to ends 321 and 325. Thus, filament 420 preferably has a round-flat-round-flat-round configuration. In other embodiments, filament 400 may have configurations as described with respect to filament 320. For example, filament 400 may have a round-flat-round, round-flat, flat-round, rectangular-flat-rectangular, entirely flat, or any other configuration as desired.

In addition, tape portions 423 and 425 may be joined to intermediate portion 421 and end portions 422 and 424 by being braided together as a single construct or coupled by other means such as gluing, sewing, or welding together, for example. First and second ends 422, 424 and intermediate portion 421 also have a corresponding height and width, or, alternatively, a diameter. The width of tape portions 423 and 425 are greater than the width/diameter of first and second ends 422, 424 and intermediate portion 421. When applied to tissue, tape portions 423 and 425 generally extend over soft tissue and compress the tissue against bone. The flat profile and relatively large width may facilitate a broad compressive footprint and may help reduce irritation of the tissue.

Sleeve 412 can be assembled with filament 420 in a similar fashion to fixation assembly 200, shown in FIG. 3A. For example, sleeve 412 may be positioned about a length of the intermediate portion 421 that extends between junction 427 and second tape portion 425, or, alternatively, between junction 427 and first tape portion 423. In some embodiments, depending on the configuration of filament 400, sleeve 412 may be positioned about tape portion 422 or 424.

FIGS. 5C-5E depict one exemplary method of using fixation assembly 400, which is similar to the method of using fixation device 200, as shown in FIGS. 3B-3E. In this method, a bone hole 452 may be formed in bone 450 and sleeve 412 inserted into the bone hole 450, as shown in FIG. 5C. First end 422, first tape portion 423 and loop 426 are passed through a first tissue penetration location 462, and second end 424 and second tape portion 425 are passed through a second tissue penetration location 464. Second end 424 and second tape portion 425 are then passed through loop 426 and second end 424 and optionally first end 422 are tensioned.

In an example of a rotator cuff repair, such as a partial thickness tear, as illustrated in FIGS. 5D and 5E, first and second ends 422, 424 are tensioned in a medial/lateral direction. First end 422 and first tape portion 423 are advanced over tissue 460 and anchored to bone 450 with the bone anchor 472. Second end 424 and second tape portion 425 are also advanced over tissue 460 and anchored to bone 450 with bone anchor 474. In this manner, tape portions 423 and 425 may form a broad compressive footprint to facilitate tissue adhesion to bone 450. In an alternative embodiment, both first and second ends 422, 424 may be secured laterally to bone 450 via a single bone anchor (not shown).

Alternative configurations of filament 420 and sleeve 412 and methods of using same are envisioned. For example, filament 420 and sleeve 412 can be assembled and used in a similar fashion as fixation device 100, shown in FIG. 2A.

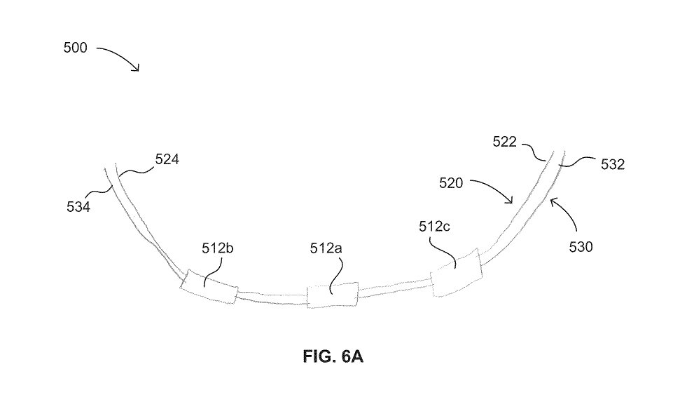

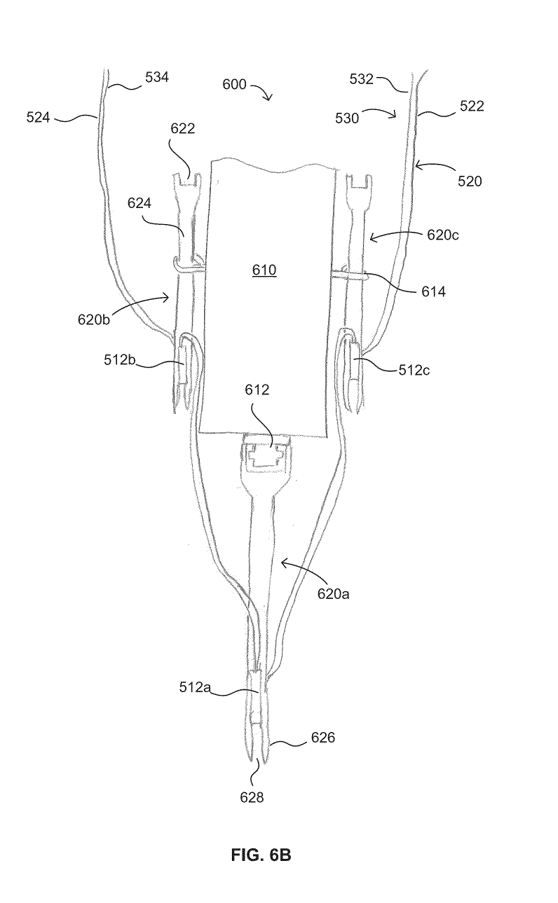

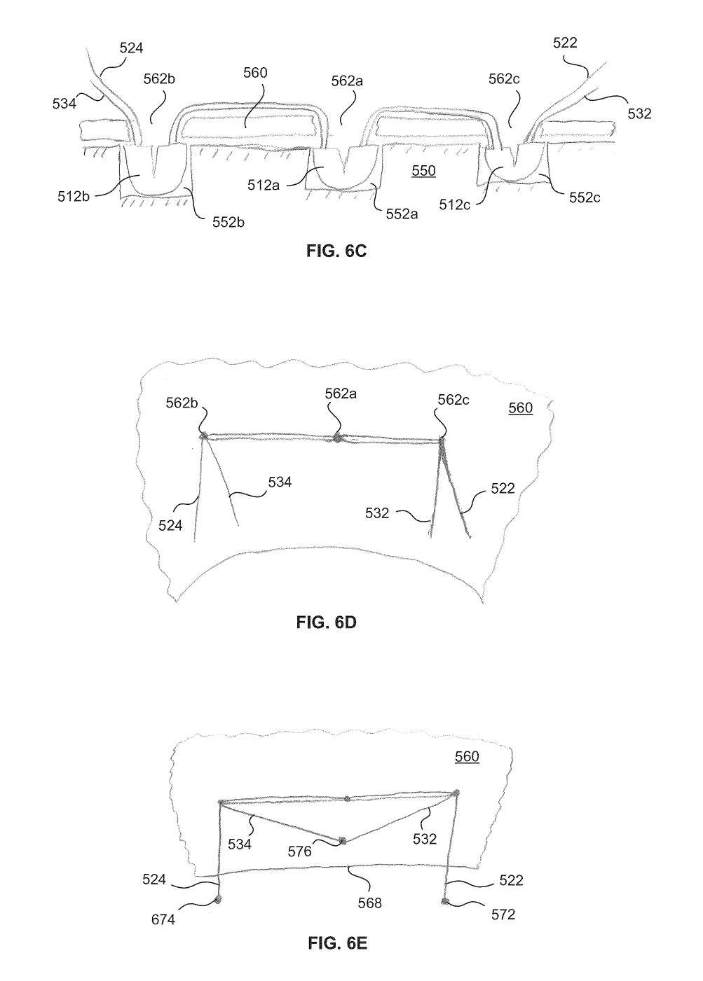

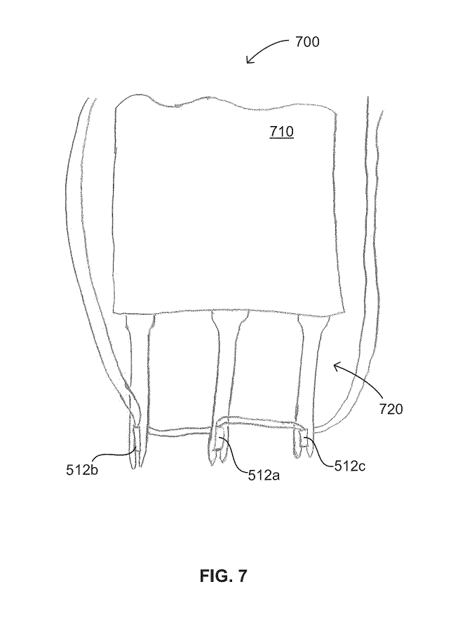

FIG. 6A depicts yet another fixation assembly embodiment 500. Fixation assembly 500 generally includes a first length of filament 520, a second length of filament 530 and three filamentary sleeves 512a-c. However, it should be understood that fixation assembly 500 can include any number of filamentary sleeves 512, such as one, two, three or four filamentary sleeves, for example. It should also be understood that any number of filaments may be utilized in fixation assembly 500, such as one, two, three, or four filaments, for example. Each of sleeves 512a-c may be the same as sleeve 12, described above. In addition, each length of filament 520, 530 may be the same as filament 20.

Continuing with the illustrated exemplary embodiment, once assembled, filaments 520 and 530 extend through each sleeve 512a-c such that first free ends 522 and 532 extend from third sleeve 512c and second free ends 524 and 534 extend from second sleeve 512b. In some embodiments, a single length of filament may be assembled with sleeves 512a-c in the same manner as filaments 20, 120, and 220 as shown in FIGS. 1A, 2A, and 3A.

FIG. 6B depicts one embodiment of an inserter device 600, which can be implemented with fixation assembly 500. Inserter 600 generally includes a body 610, a head connector 612, retaining arms 614, and three removable heads 620a-c. However, it should be understood that any number of removable heads 620 may be utilized, which may largely depend on the number of sleeves 512 being implanted. For example, inserter device 600 may include one, two, three, or four removable heads 620.

Removable heads 620a-c each generally include a connector portion 622, an elongate shaft 624, and an insertion tip 626 having a retaining slot 628. Elongate shaft 624 may be sufficiently long to be implemented through an arthroscopic cannula. Each head 620 is capable of being attached and detached to the connector 612 via a quick-connect mechanism, which may include magnets, a ball detent, or the like. Insertion tip 626 may be sharpened to penetrate tissue and insert sleeve 512 into a preformed bone hole. In other embodiments, penetration end 626 may be sharpened to penetrate bone and tissue in the manner of a punch. Retaining slot 628 is configured to releasably hold sleeve 512 in a bent configuration while filaments 520 and 530 are slidably retained by each sleeve 512a-c. Optionally, an actuating arm or arms (not shown) can cover slot 628 during penetration of tissue and can be actuated so that it is moved out of the way during implantation of sleeve into bone. Retaining members 614 are attached to body 610 and configured to hold any of the removable heads 620a-c.

Inserter 600 and fixation assembly 500 may be preassembled, packaged, and delivered to the operating theater. Alternatively, inserter 600 and fixation assembly 500 may be packaged and delivered unassembled to the operating theater where assembly takes place. When assembled for use, first removable head 620a is attached to connector 612 and second and third removable heads 620b, 620c are retained by retaining members 614. Each head 620a-c includes a respective sleeve 512 located in respective slots 628 and each filament 520, 530 is disposed within each sleeve 512a-c such that first free ends 522 and 532 and second free ends 524 and 534 extend from removable heads 620c and 620b, respectively. Filaments 520 and 530 are slidable within sleeves 512a-c so that they may be tensioned during implantation of sleeves 512a-c as needed.

FIGS. 6C-6E depict one exemplary embodiment of a method of using inserter device 600 and fixation assembly 500. In this method, each sleeve 512a-c is generally inserted through tissue 560 and implanted into bone 550. However, it is envisioned that each sleeve 512a-c may be implanted into bone 550 and then a single length of filament having a loop may be passed through tissue 560 in a similar manner as that described with respect to FIGS. 1A-5E.

Prior to implantation, three bone holes 552a-c, one for each sleeve 512a-c, may be formed in bone 550 at desired locations. For example, in a rotator cuff reparation procedure, bone holes 552a-c may be formed in a medial row generally aligned in an anterior/posterior direction. Tissue 560 may then be tensioned and first head 620a containing first sleeve 512a is inserted through tissue 560 at first tissue penetration location 562a. Thereafter, insertion tip 626 and sleeve 512a are inserted into the first bone hole 552a, sleeve 512a is released therein, and head 620a is removed from the bone hole 552a. Filaments 520 and 530, which extend from first bone hole 552a, first penetration 562a, and through sleeves 512b and 512c, are tensioned to fully seat sleeve 512a.

Thereafter, first head 620a is detached from connector 612 and second head 620b retaining second sleeve 512b is attached to connector 612. Second head 620b is then inserted through tissue 560 at a second tissue penetration location 562b. Second sleeve 512b is inserted into second bone hole 552b and released therein. Second head 620b is removed from second bone hole 552b and second free ends 524 and 534 along with a portion of filaments 520 and 530 that extend between the first and second sleeves 512a, 512b are tensioned to fully seat sleeve 512b.

Thereafter, second head 620b is detached from connector 612 and third head 620c retaining third sleeve 512c is attached to connector 612. Third head 620c is then inserted through tissue 560 at a third tissue penetration location 562c. Third sleeve 512c is inserted into third bone hole 552c and released therein. Third head 620c is removed from third bone hole 552c and first free ends 522 and 532 along with a portion of filaments 520 and 530 that extend between the first and third sleeves 512a, 512c are tensioned to fully seat third sleeve 512c.

The operator retains control of first free ends 522 and 532 and second free ends 524 and 534. As illustrated in FIGS. 6D and 6E, these ends are then tensioned which cinches down the portions of filaments 520 and 530 that extend between each sleeve 512a-c. As this occurs, tissue 560 underlying these portions of filaments 520 and 530 is compressed against the underlying bone. The free ends 522, 524, 532, 534 are available to be attached to one or more bone anchors, filamentary or the like. For example, in a rotator cuff repair and as shown in FIG. 6E, first free end 522 and second free end 524 may be anchored via anchors 572 and 574, respectively, to the humerus beyond the lateral edge 568 of the tissue 560. Additionally, first free end 532 and second free end 534 may be anchored to the humerus through tissue 560 via anchor 576, as shown. While FIGS. 6D and 6E illustrate one example, other configurations may be formed dependent on the type of soft tissue, type of repair, number of filaments, and number of bone anchors.

FIG. 7 depicts an alternative inserter device 700, which may be utilized in conjunction with fixation assembly 500. Inserter device 700 is similar to inserter device 600 in that it includes a body 710 and a plurality of heads 720. In addition, each head 720 releasably retains a filamentary sleeve 512 while at least one filament extends through each sleeve. However, unlike inserter 600, each head 720 is attached to body 710 in a configuration for substantially simultaneous insertion of sleeves 512a-c. Thus, during operation, each head 512a-c concurrently punctures through tissue 560 and is advanced into their respective bone holes where sleeves 512a-c are deposited and anchored. It is envisioned that the body 710 could be adjusted, or otherwise, to adjust the spacing of the heads 720.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of tissue fixation comprising:
inserting an anchor into a bone hole, the anchor having a length of filament slidably disposed therein, the length of filament being comprised of a first portion and a second portion, the first portion having a cross-sectional dimension larger than a cross-sectional dimension of the second portion, the second portion forming a loop;
passing the first portion through tissue at a first location and the loop at least partially through tissue at a second location; and
advancing the first portion through the loop.

2. The method of claim 1, wherein the anchor is a filamentary sleeve having a first opening and a second opening, the first portion extending from the first opening and the loop extending from the second opening.

3. The method of claim 1, wherein the loop is formed by splicing the single length of filament with itself at a location along its length.

4. The method of claim 1, wherein the first portion includes a flat cross-sectional profile having a width and a height, and the second portion includes a round cross-sectional profile having a diameter.

5. The method of claim 1, wherein the first portion includes a flat cross-sectional profile having a width and a height, and the second portion includes a rectangular cross-sectional profile having a width and a height.

6. The method of claim 1, further comprising:
passing a third portion of the length of filament through the tissue at the second location, the third portion extending from a crotch of the loop and having a cross-sectional dimension larger than the cross-sectional dimension of the second portion.

7. The method of claim 1, further comprising:
passing a third portion of the length of filament through the tissue at a third location, the third portion having a cross-sectional dimension larger than the cross-sectional dimension of the second portion.

8. The method of claim 7, further comprising:
advancing the third portion through the loop.

9. A method of tissue fixation comprising:
obtaining an insertion device having a head connector, a first head having a first filamentary sleeve coupled thereto, a second head having a second filamentary sleeve coupled thereto, and a single length of filament extending through the first and second sleeves;
inserting the first head and first filamentary sleeve into bone at a first location;
with the first sleeve remaining in the first location, removing the first head from the bone;
removing the first head from the head connector;
attaching the second head to the head connector;
inserting the second head and second filamentary sleeve into the bone at a second location; and
removing the second head from the second head connector,
wherein the first and second filamentary sleeves disposed in the bone and the single length of filament is disposed at least partially within the first and second filamentary sleeves.

10. The method of claim 9, wherein a first free end of the single length of filament extends from the first filamentary sleeve and a second free end of the single length of filament extends from the second filamentary sleeve.

11. The method of claim 10, wherein the inserting step includes inserting the first and second heads through tissue such that a segment of the single length of filament extending between the first and second sleeves compresses the tissue against the bone.

12. The method of claim 11, wherein inserting the first and second filamentary sleeves occurs concurrently.

13. The method of claim 9, wherein first and second free ends of the single length of filament extend from the first filamentary sleeve and a loop-end of the single length of filament extends from the second filamentary sleeve.

14. The method of claim 13, further comprising:
passing the first and second free ends through tissue at first and second locations respectively;
passing the loop-end through the tissue at a third location; and
advancing the first and second free ends through a loop defined by the loop-end.

15. A method of tissue fixation comprising:
inserting a first head of an insertion device through tissue and into a first bone hole, the first head being attached to a connector and having a first sleeve attached thereto, the insertion device also having a second head having a second sleeve attached thereto and a third head having a third sleeve attached thereto, a length of filament is slidably disposed through the first, second, and third sleeves;
securing the first sleeve within the first bone hole while the length of filament remains slidably disposed within the first, second, and third sleeves;
removing the first head from a connector;
attaching the second head to the connector;
inserting the second head through the tissue and into a second bone hole; and
securing the second sleeve within the second bone hole.

16. The method of claim 15, further comprising:
removing the second head from the connector;
attaching the third head to the connector;
inserting the third head through tissue into a third bone hole; and
securing the third sleeve within the third bone hole.

17. The method of claim 16, further comprising:
tensioning the length of filament;
connecting an end of the length of filament to a bone anchor; and
anchoring the bone anchor into the bone.
